(19) United States
(12) Patent Application Publication
(10) Pub. No.: US 2007/0213668 A1
(43) Pub. Date: Sep. 13, 2007

(54) Foot Operated Irrigation Control Apparatus for Medical Procedures

(76) Inventor: Aaron Spitz, Laguna Beach, CA (US)

Correspondence Address: CHRISTIE, PARKER & HALE, LLP, PO BOX 7068, PASADENA, CA, 91109-7068, US

(21) Appl. No.: 11/683,421

(22) Filed: Mar. 7, 2007

Related U.S. Application Data
(60) Provisional application No. 60/780,452, filed on Mar. 7, 2006.

(51) Int. Cl. A61M 37/00

(52) U.S. Cl. 604/131

(57) ABSTRACT

A foot operated irrigation pump and flow control device for controlling the flow of irrigation fluid to a medical instrument. The device includes a flow control unit that is operable by an operator's foot to control a flow rate of fluid through a passageway therethrough, and a pump unit that is in fluid connection with the flow control unit and which is operable by an operator's foot to selective pump fluid out of the pump unit.

### CROSS-REFERENCE TO RELATED APPLICATIONS

This application claims priority to and the benefit of U.S. Provisional Patent Application No. 60/780,452, filed on Mar. 7, 2006, the entire content of which is incorporated by reference herein.

### SUMMARY OF THE INVENTION

The invention relates to a device for regulating the inflow of irrigation fluids during endoscopy procedures, and more particularly to a device that includes one or more foot operated flow regulators that allow the operator to keep his or her hands free to manipulate the scope or other instruments and devices, and also eliminates the need for the use of the physician's hands or those of an assistant to regulate the irrigation inflow as is commonly required with hand controlled irrigation regulators.

### BACKGROUND

A physician my use an endoscope to access, visualize and treat disorders of the urinary tract. When used to view the ureters (the tubular structures that carry urine from the kidneys to the bladder), the endoscope is called an ureteroscope. When used to view the urethra (a tubular structure that carries urine from the bladder to the outside of the body), the endoscope is called a cystoscope. When used to view the inner lining of the kidney directly, the endoscope is called a nephroscope. These scopes utilize optical fibers or rigid lens systems for carrying an image from the distal lighted tip of the instrument to a viewing piece at the proximal end. Many scopes have extra lumens to guide other instruments for procedures to treat urinary problems. As a partial listing, physicians can choose to use cystoscopy for examination of the following types of conditions: frequent urinary tract infections; blood in the urine (hematuria); loss of bladder control (incontinence) or overactive bladder; unusual cells found in urine samples; urinary retention; painful urination, chronic pelvic pain, or interstitial cystitis; urinary blockage such as prostate enlargement, stricture, or narrowing of the urinary tract; stone in the urinary tract; and unusual growth, polyp, tumor, or cancer.

For example, if a patient has a calcification lodged proximally in the urinary tract, the treating physician may extend the ureteroscope through the bladder and up into the ureter. The physician can then visualize the stone and remove it with a small basket at the end of a wire inserted through an extra lumen in the ureteroscope used for that purpose. The physician may also use the extra lumen in the cystoscope to extend a flexible fiber that carries a laser beam to break the stone into smaller pieces that can then be passed out of the body though urination. In any of these procedures, and many others, irrigation fluid is passed through the cystoscope to aid in visualization.

As can be appreciated, ureteroscopy and cystoscopy are exceedingly valuable procedures carried out by physicians, including urologists. However, when using ureteroscopes and cystoscopes, the physician's hands, or those of his assistant, are often occupied with the operation of regulating the flow of irrigation fluid to a degree that is detracting from performing other tasks simultaneously which would otherwise require the manual dexterity being dedicated toward irrigation control. Therefore, during ureteroscopy and cystoscopy, the urologist is provided with an assistant. This requires both communication and coordination between the urologist and the assistant, which can be distracting to the physician or inadequately rendered. Moreover, the additional assistance required may raise the costs of performing these urological procedures. There accordingly remains a need for an improved irrigation control device that can be fully operated by a single person utilizing a foot control so as to leave free the more critical use of one's hands, whether it is the physician's hands or an assistant's hands.

### BRIEF DESCRIPTION OF THE DRAWINGS

FIG. 1 is a diagrammatic view showing a layout of an exemplary foot operated irrigation pump and flow control device connected to a fluid source and an ureteroscope or cystoscope.

FIG. 2 is a top plan view of an exemplary embodiment of the irrigation flow control and fluid pump components of the invention.

FIG. 3 is a top isometric view of an exemplary irrigation flow control and fluid pump units of the invention shown separated.

FIG. 3A is a side view of a swinging clip for holding tubing in place on the platforms of the device.

FIGS. 3B and 3C are isometric views of a squeeze bulb support.

FIG. 4 is an isometric view of the exemplary irrigation flow control unit of FIGS. 1 and 2.

FIG. 5 is a longitudinal cross-section view of the exemplary irrigation flow control unit of FIG. 4.

FIG. 6 is a top plan view of the exemplary irrigation flow control unit of FIG. 5 with its roller housing removed.

FIG. 7 is a perspective view of another exemplary irrigation flow control unit with open and closed modes, shown without tubing and in its open mode.

FIG. 8 is a perspective view of the exemplary irrigation flow control unit of FIG. 8 in its closed mode with tubing passing therethrough being clamped closed.

FIG. 9 is a perspective view of another exemplary irrigation flow control unit with multiple degrees of ratcheting, shown without tubing and in its open mode.

FIG. 10 is a front isometric view of another exemplary foot squeeze bulb of the irrigation control device of the invention.

### DETAILED DESCRIPTION OF THE INVENTION

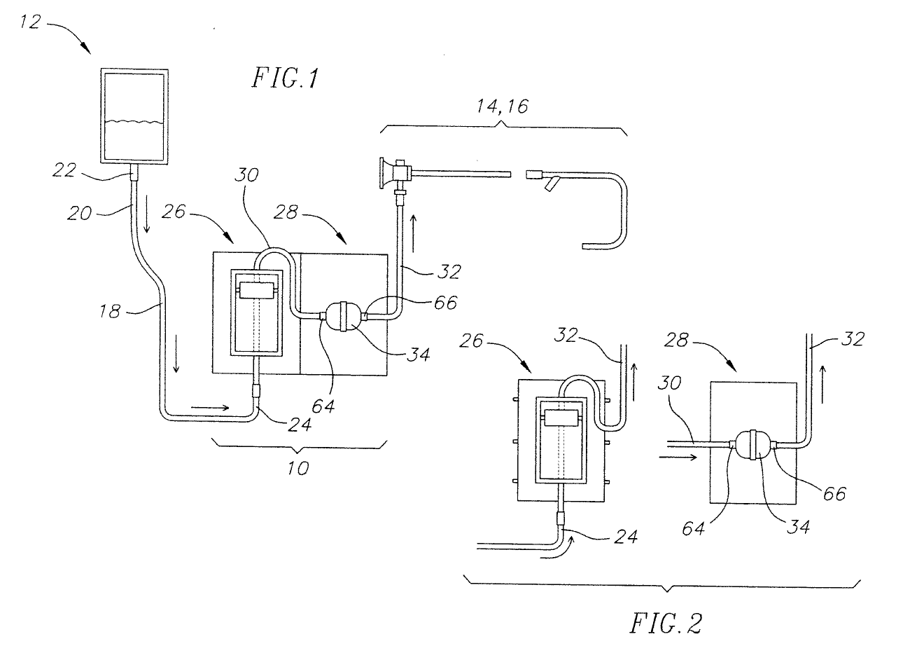

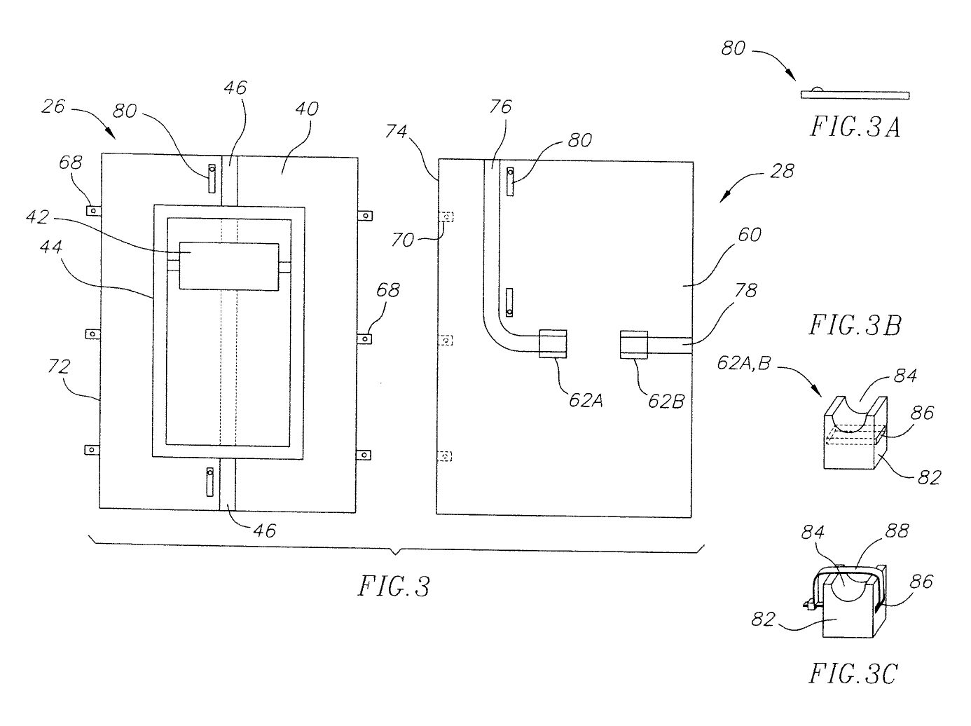

FIG. 1 is a diagrammatic view showing a layout of an exemplary foot operated irrigation pump and flow control device 10 connected to a fluid source 12 and a rigid or flexible ureteroscope 14 or a rigid or flexible cystoscope 16. The fluid source 12 can comprise conventional bottles or bags of irrigation fluid, such as saline solution. The fluid source 12 is preferably elevated (e.g., a few feet) above to the level of the ureteroscope 14 or the cystoscope 16 during use on a patient (who is positioned in dorsal lithotomy position on his/her back) so that there will be a positive pressure on the irrigating fluid to cause it to flow out of the ureteroscope 14 or the cystoscope 16. In lieu of elevating the fluid source 12 relative to the level of the patient to create positive pressure, other means can be used to create a positive pressure of the fluid, such as a pump (not shown.) A fluid line 18 is connected at a first end 20 to an outlet end 22 of the fluid source 12. The other end 24 of the fluid line 18 is connected to either a flow rate control unit 26 that connects to a fluid pump unit 28 that in turn is connected to the ureteroscope 14 or the cystoscope 16, or the flow rate control unit 26 or the fluid pump unit 28 is directly connected to the ureteroscope 14 or the cystoscope 16 by an outlet tube 32, as shown in FIG. 2. The foot operated irrigation pump and flow control device 10 is preferably positioned on the floor where the surgeon can easily reach and manipulate it with his or her foot. A short length of tubing 30 passes through the flow rate control unit 26 and connects to a compressible bladder or squeeze bulb 34 that is mounted on the fluid pump unit 28, and is in fluid communication with the outlet tube 32 that connects to the ureteroscope 14 or the cystoscope 16. The compressible bladder or squeeze bulb 34 is preferably formed in whole or in part of transparent material to allow the person setting up the device to quickly visually determine if all air bubbles are out of the system prior to use. Ensuring that all air is out of the system is helpful since air in the system can impair visibility during irrigation. The squeeze bulb 34 is also preferably constructed so that it has sufficiently great memory to re-expand itself rapidly so as to minimize the decrease in flow rate through the device while it is re-expanding and to minimize the time the surgeon must wait before he or she can recompress the device. If the material used to make the walls of the squeeze bulb 34, e.g., silicone rubber, does not itself have sufficient memory, then a mechanical device, such as a spring placed inside the squeeze bulb (not shown) can be used to help re-expand the squeeze bulb quickly and fully after squeezing pressure is released. Preferably, the flow rate through the foot operated irrigation pump and flow control device 10, including the tubing leading to and from the device, should be minimally impeding so that with the device 10 in line with the fluid source 14 and the operating scope (e.g., the ureteroscope 14 or the cystoscope 16), the flow rate is minimally or not at all decreased to the operating scope compared to the flow rate of the fluid to the operating scope when no device 10 is in use, unless the user decides to limit or shut off the flow. This may be accomplished by ensuring that any connections and valves have sufficiently large cross-sectional areas, flow paths and mechanics to minimize reduction in flow rates at these junctions.

FIG. 2 is a top plan view of an exemplary embodiment of the irrigation flow control unit 26 and the fluid pump unit 28 shown in FIG. 1, but separated. As can be seen, the flow rate control unit 26 either connects to the fluid pump unit 28 by the short length of tubing 30, which fluid pump component 28 is in turn connected to the ureteroscope or the cystoscope (not shown), or directly connects with the ureteroscope or the cystoscope by the outlet tube 32. The irrigation flow control unit 26 and the fluid pump unit 28 can be used together or individually. In other words, it is possible to include one or both of the functionalities (e.g. flow control and/or pumping) in the device.

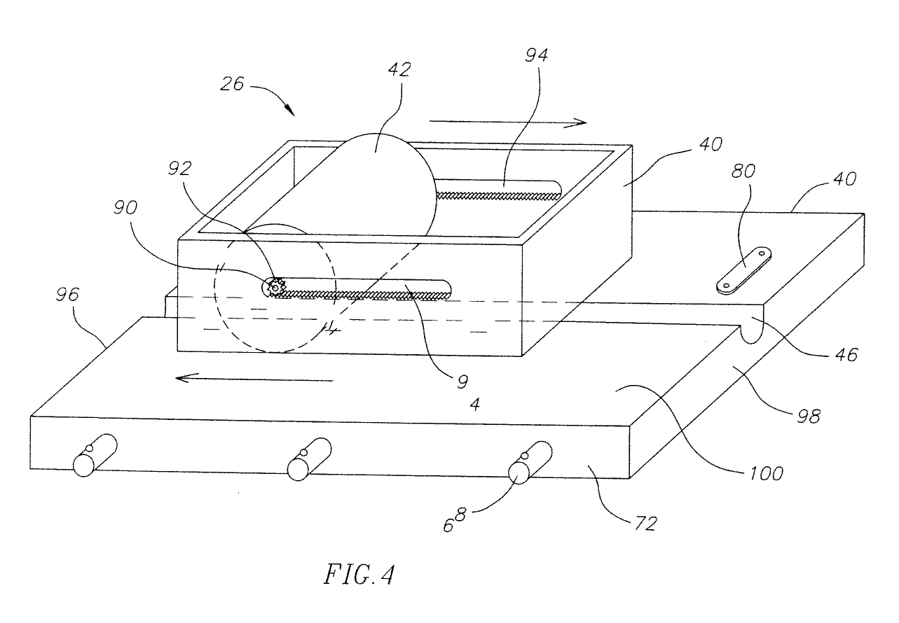

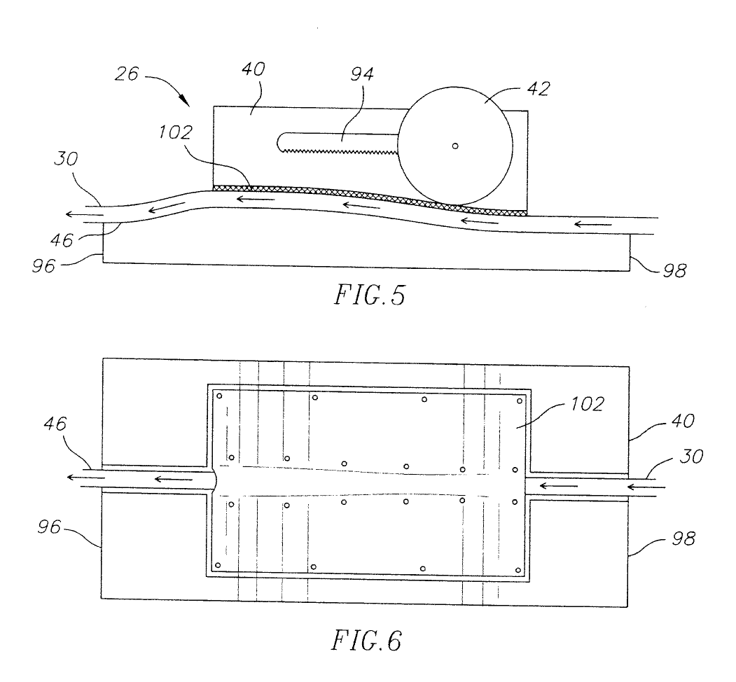

FIG. 3 is a top isometric view of the exemplary irrigation flow control unit 26 and the fluid pump control unit 28 of the invention. The irrigation flow control unit 26 has a platform 40. A roller 42 is located in a roller housing 44. Tubing will run along a tubing route 46 (e.g., a channel) on the platform 40 and the tubing can comprise a length of silicone rubber (e.g. Silastic®) or other plastic or rubber tubing. The roller 42 and housing 44 arrangement is designed with respect to the platform and tubing such that the action of advancing the roller 42 in one direction compresses the tubing onto the platform, thus restricting the flow of liquid through the tubing or halting it altogether, while advancing the roller 42 in the other direction will release compression of the tubing and thus not restrict fluid flow therethrough. On the right side there is shown the fluid pump control unit 28. It includes a platform 60 with two bulb supports 62A, 62B onto which is mounted a squeeze bulb 34 (see FIGS. 1 and 2) is positioned. The squeeze bulb 34 has a one way valves (not shown) that allows for inflow through an ingress side 64 of the squeeze bulb 34 and outflow through an egress side 66 of the squeeze bulb 34, such that compression on the squeeze bulb 34 propels fluid with increased flow toward the endoscope or the cystoscope, and relaxation on the squeeze bulb 34 after a compression creates negative pressure within the bulb that draws fluid into it from the ingress side 64 from the fluid source 12. This squeeze bulb 34 can be of convention design. The platform 60 includes tubing routes 76 and 78, which can comprise grooves or other structures or mechanism for retaining the tubing on the platform 60. The order of the exemplary irrigation flow control unit 26 and the fluid pump control unit 28 can be reversed if desired, e.g., if the device is positioned on the contra lateral side of the surgeon in an inverse orientation. Together these two units 26 and 28 of the device 10 give the surgeon the ability to turn off, slow down or increase the flow rate of irrigation fluid through the endoscope or cystoscope. The irrigation flow control unit 26 and the fluid pump control unit 28 optionally have connectors 68 and 70, respectively, on their side 72 and 74, respectively, which connectors 68 and 70 are aligned and adapted to engage together to hold the irrigation flow control unit 26 and the fluid pump control unit 28. For example, the connections 68 can comprises prongs with spring loaded clips and the connectors 70 can comprises holes adapted to releasably receive the prongs 68. Other types of connectors can be used as desired. The two units 26 and 28 can also be integral if desired.

Detail FIG. 3A shows a swing clip 80 which is adapted to releasably retain tubing in place in grooves 46, and 76 and 78 formed on the platforms 40 and 50, respectively. This feature permits the tubing to be discarded after use, and reuse of the platforms and other components to save costs.

Detail FIGS. 3B and 3C show and exemplary embodiment of the two bulb supports 62A, 62B, which each comprise an uplifted platform 82 with a recess 84 formed at its top. The recesses 84 are preferably sized and shaped to retain the ingress and egress sides 64 and 66, respectively, of the squeeze bulb (as shown in FIG. 1), and a slot 86 is formed beneath the recess 84 for receiving a synch tie 88 therethrough for retaining the squeeze bulb in place. Other types of restraints can be used to hold the squeeze bulb in place. Again, the squeeze bulb and its associated tubing can be discarded after use.

FIG. 4 is an isometric view of the exemplary irrigation flow control unit 26 of FIGS. 1-3. As is shown, the platform 40 of the irrigation flow control unit 26 has its channel 46 formed in the top thereof, varying in depth in a ramp-like arrangement, so that rolling the roller 42 in the roller housing 40 will differentially impinge (or not impinge) on the tubing (not shown) located in the channel 46. The roller 42 has an axle 90 with a gear 92. The roller housing 40 has geared slots 94 on which move the toothed gear 90. When the roller 42 is rolled forwardly towards one end 96 of the housing 44 where the channel 46 is at its shallowest relative to the top surface 100 of the platform 40, the roller 42 will impinge on the tubing to a greater degree to partially or completely close it off so that no fluid can flow therethrough. By rolling the roller 42 towards end 98 where the channel 46 is deepest relative to the top surface 100 of the platform 40, the roller 46 will not impinge on the tubing, and thus will not stop or slow fluid flow therethrough. The prongs 68 are also shown extending from side walls 72 of the platform 40.

FIG. 5 is a longitudinal cross-section view of the exemplary irrigation flow control unit 26 of FIG. 5, showing tubing 30 passing through the channel 46. As can be appreciated, by rolling roller 42 along slot 94 towards first end 96, the roller will squeeze down on the tubing 30 and partially or completely close it off. If desired, to provide added protection for the tubing 30, a pliable layer 102 can overlay the tubing 30 placed in the channel 46. This pliable layer 102 be peeled back when the tubing 30 is to be replaced. For this purpose, the roller housing 40 can be detachably attachable to the platform 40, such as by being hingeably attached at one side, or attached by clips or other known means (not shown.) Other mechanism can be used if desired.

FIG. 6 is a top plan view of the exemplary irrigation flow control unit 26 of FIG. 6 with its roller and roller housing 40 shown removed, to show the pliable layer 102 overlaying the tubing 30 placed in the channel 46.

In lieu of providing the exemplary irrigation flow control unit 26 as having a rolling wheel that has graduations to control the flow therethrough from a slow flow to a fast flow, it is also possible to provide an irrigation flow control unit that has two positions, either on, wherein the fluid flow therethrough is unimpeded, or off, wherein no fluid will flow through the irrigation flow control unit.

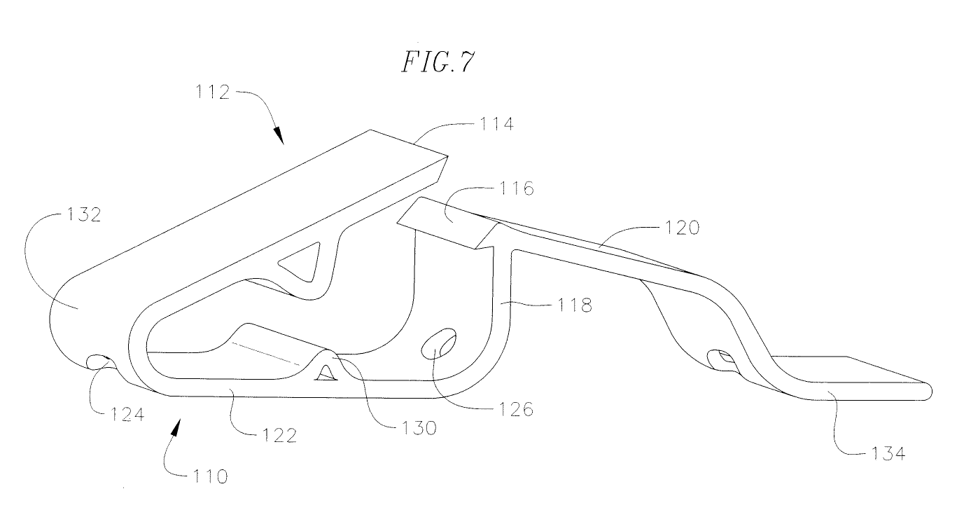

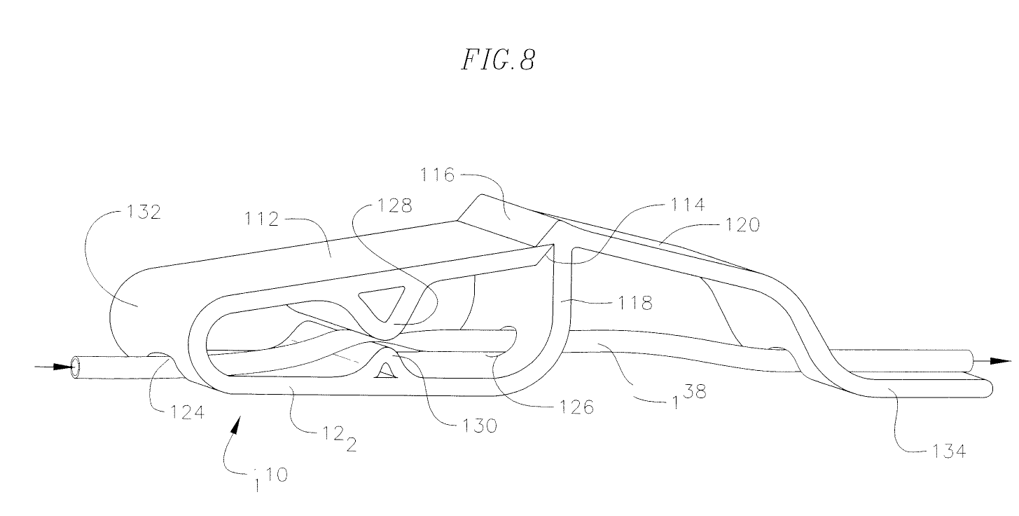

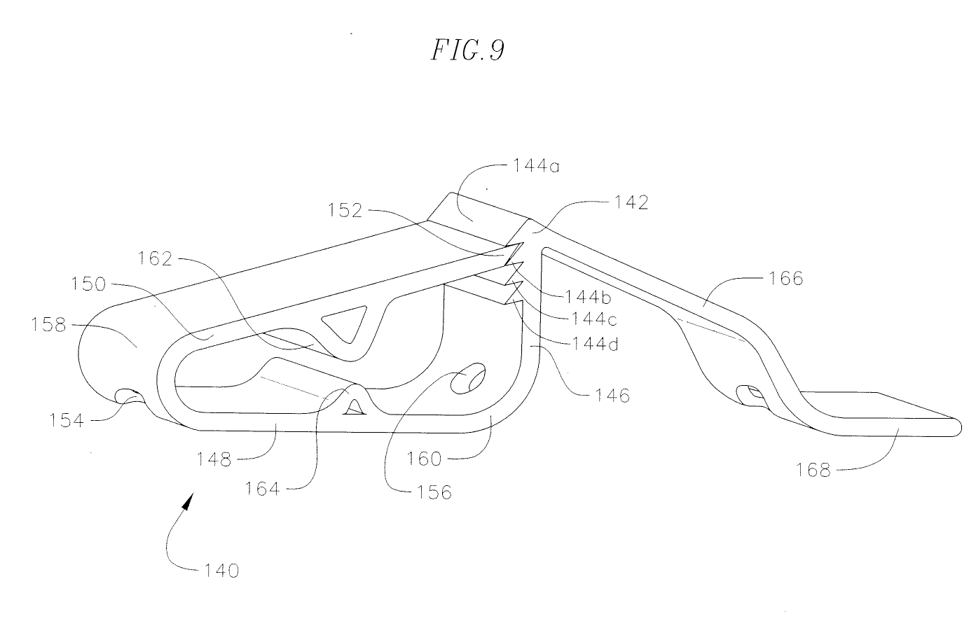

FIG. 7 is a perspective view of an exemplary irrigation flow control unit 110 with open and closed modes, shown without a tube and in its open mode and FIG. 8 is a perspective view of the exemplary irrigation flow control unit 110 of FIG. 8 in its closed mode with a tube 138 passing therethrough being clamped closed. The irrigation flow control unit 110 has a locking lever 112 with a free end 114 that is adapted to be captured and locked in place by a catch 116. The catch 116 can be continuous with a catch wall 118 and an opening lever 120. The catch wall 118 continues from a base 122 that can be rested on a floor surface. Tube passageways 124 and 126 are formed through a front area 132 and a transition area of the catch wall 118 and the base 122, respectively, and provide a passageway for a tube 138 to pass therethrough. The pinching protrusions 128 and 130 extend inwardly from the locking lever 112 and the base 122, respectively, and are provided to impinge on the tube 138, when the locking lever 112 is pushed down (e.g. by a user's foot) so that the free end 114 of the locking lever 112 is caught by the catch 116. When a user wishes to release the pinching force on the tube 140, the user will depress the opening lever 120, e.g., by stepping on it, which causes the catch wall 118 to arch back rearwardly and release the free end 114 of the locking lever 112 from the catch 116. The base 122 and a free end 134 of the opening lever 120 will preferably rest on a floor surface and can be made wide enough to support the irrigation flow control unit 110 there. Alternately, stabilizing devices or means, such as suction cups, non-skip material, etc. can be provided for stabilization purposes. For low cost, preferably the irrigation flow control unit 110 can be formed as a single plastic unit.

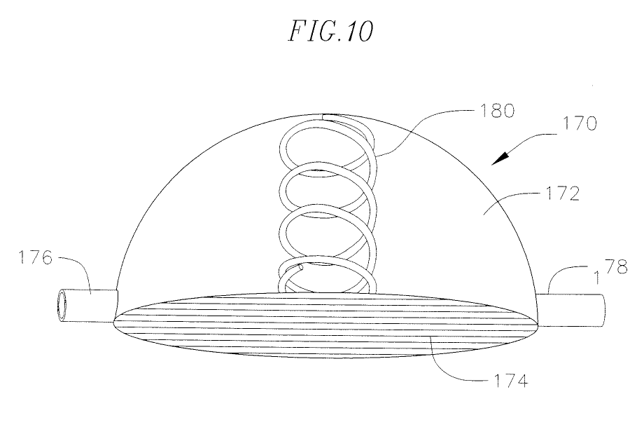

FIG. 9 is a perspective view of another exemplary irrigation flow control unit 140, shown without tubing and in its open mode, is similar to the irrigation flow control unit 110 shown in FIGS. 7 and 8, except that it has multiple degrees of ratcheting to provide for addition degrees of flow control and flow stoppage through a tube passing therethough. In this irrigation flow control unit 140, a catch 142 has multiple protrusions 144a, 144b, 144c, and 144d, which provide for varying degrees of compression. Although four protrusions are shown, fewer or more can be provided. The protrusions 144a, 144b, 144c, and 144d are formed on catch wall 146, which extends from a base 148 that can be rested on a floor surface. A locking lever 150 is provided and has a free end 152. Tube passageways 154 and 156 are formed through a front area 158 and a transition area 160 of the catch wall 146 and the base 148, respectively, and provide a passageway for a tube (not shown) to pass therethrough. Pinching protrusions 162 and 164 extend inwardly from the locking lever 150 and the base 148, respectively, and provide varying degrees of impingement on the tube (such as partially blocking of the tube to complete blocking of the tube so no fluid can flow therethrough) when the locking lever 150 is pushed down (e.g. by a user's foot) so that the free end 152 of the locking lever 150 will be caught by one of the protrusions 144a, 144b, 144c or 144d of the catch 142. When a user wishes to release the pinching force on the tube, the user will depress an opening lever 166, e.g., by stepping on it, which causes the catch wall 146 to arch back rearwardly and release the free end 152 of the locking lever 150 from the catch 142. As with the embodiment shown in FIGS. 7 and 8, the base 148 and/or a free end 168 of the opening lever 120 will preferably rest on a floor surface and can be made wide enough to support the irrigation flow control unit 140 there. Alternately, stabilizing devices or means, such as suction cups, non-skip material, etc. can be provided for stabilization purposes. For low cost, preferably the irrigation flow control unit 110 can be formed as a single plastic unit.

The irrigation flow control units 26/110/140 and the fluid pump control unit 28 shown and described above are exemplary embodiments of these units, and other mechanism can be used to provide the functionality that these units provide. For example, in lieu of the above described mechanism to controls the irrigation fluid flow rate by pinching off the irrigation fluid supply line by a rolling mechanism, it is also feasible, to use a foot operated mechanical or electric flow control valve or some other device. Additionally, while using a squeeze bulb 28 in the fluid pump control unit 28 is a simple and low cost approach, other types of mechanical pumps or even electronics pumps that can be controlled by a user's foot motion can also be used. An important aspect of the invention is the operability of the irrigation flow control units 26/110/140 and the fluid pump control unit 28 by a user's foot action, thereby freeing the user's hand for other purposes. Another aspect of the invention is the separability of the irrigation flow control units 26/110/140 and the fluid pump control unit 28 that that one or both units can be operated by a single user.

FIG. 10 is a front isometric view of another exemplary foot squeeze bulb 170 for the irrigation control device of the invention. In this embodiment of a squeeze bulb 170, there is shown a generally semi-spherical upper portion 172 and a flattened lower portion 174, with a fluid inlet 176 and a fluid outlet 178 connected thereto. However, other shapes can be used. The flattened lower portion 174 can be placed on a floor surface or a platform and supported thereby. The user will then step on the upper portion 172 to provide the motive force to move fluid through the squeeze bulb 170. Unlike the squeeze bulb 34 of FIGS. 1 and 2, which requires brackets to hold it in place, this squeeze bulb 170 with a flattened bottom will naturally stay in position during the squeezing process. This squeeze bulb 170 will include a one-way valve (not shown) to ensure fluid only flows out of fluid outlet 178. The foot squeeze bulb 170, like the squeeze bulb 34 shown in FIG. 1, is preferably formed in whole or in part of transparent material to allow the person setting up the device to quickly visually determine if all air bubbles are out of the system prior to use. The squeeze bulb 170 is also preferably constructed so that it has sufficiently great memory to re-expand itself rapidly so as to minimize the decrease in flow rate through the device while it is re-expanding and to minimize the time the surgeon must wait before he or she can recompress the device. If the material used to make the walls of the squeeze bulb 34, e.g., silicone rubber does not itself have sufficient memory, then a mechanical device, such as a spring 180 placed inside the squeeze bulb 170 can be used to help re-expand the squeeze bulb quickly and fully after squeezing pressure is released.

Although embodiments of the present invention have been described in detail hereinabove in connection with certain exemplary embodiments, it should be understood that the invention is not limited to the disclosed exemplary embodiments, but, on the contrary is intended to cover various modifications and/or equivalent arrangements included within the spirit and scope of the present invention as set forth in the appended claims.

What is claimed is:

1. A foot operated irrigation pump and flow control device for controlling the flow of irrigation fluid to a medical instrument, comprising:
a flow control unit that is operable by an operator's foot to control a flow rate of fluid through a passageway therethrough; and
a pump unit that is in fluid connection with the flow control unit and which is operable by an operator's foot to selective pump fluid out of the pump unit.

2. The foot operated irrigation pump and flow control device of claim 1, wherein the flow control unit comprises a roller, a roller housing, and a platform, and wherein the flow control unit is adapted to be placed on a floor surface so that the roller can be manipulated by a user's foot, and depending on which direction the roller is rolled, it will selectively open and close off the passageway to control the flow of fluid through the passageway.

3. The foot operated irrigation pump and flow control device of claim 1, wherein the pump unit comprises a platform and a squeeze bulb, the pump unit being adapted to be placed on a floor surface and compressed by a user's foot to pump fluid therethrough.

4. The foot operated irrigation pump and flow control device of claim 3, wherein the passageway through the flow control unit comprises tubing connected at one end to a fluid supply, and which is connected at another end to an ingress end of the squeeze bulb.

5. The foot operated irrigation pump and flow control device of claim 1, wherein the flow control unit and the pump unit each have connectors for connecting the flow control unit and the pump unit side-by-side.

6. The foot operated irrigation pump and flow control device of claim 1, wherein one of the flow control unit and the pump unit connects to cystoscope or ureteroscope via tubing, and the other of the flow control unit and the pump unit connects to a fluid supply.

7. The foot operated irrigation pump and flow control device of claim 1, wherein the pump unit comprises a squeeze bulb that is formed in whole or in part of transparent material.

8. The foot operated irrigation pump and flow control device of claim 1, wherein the foot operated irrigation pump and flow control device can be used with irrigation from gravity and/or irrigation from under external pressure from another pump.

9. The foot operated irrigation pump and flow control device of claim 1, wherein the flow control unit has two positions, either on, wherein the fluid flow therethrough is unimpeded, or off, wherein no fluid will flow through the irrigation flow control unit.

10. The foot operated irrigation pump and flow control device of claim 9, wherein the flow control unit comprises a clamp that catches and releases in an on/off fashion with foot compression.

11. The foot operated irrigation pump and flow control device of claim 1, wherein the pump unit comprises a squeeze bulb that includes an internal spring to aid in re-expanding the squeeze bulb when external foot pressure is removed.

12. The foot operated irrigation pump and flow control device of claim 1, wherein the flow control unit and the pump unit are adapted to minimally or not at all impede with fluid flow therethrough when the flow control unit is in an open position.

13. A foot operated irrigation pump and flow control device for controlling the flow of irrigation fluid to a medical instrument, comprising:
a flow control unit that is operable by an operator's foot to turn on or off fluid flow through a passageway therethrough; and
a pump unit that is in fluid connection with the flow control unit and which is operable by an operator's foot to selective pump fluid out of the pump unit.

14. The foot operated irrigation pump and flow control device of claim 13, wherein the pump comprises a squeeze bulb that is formed in whole or in part of transparent material.

15. The foot operated irrigation pump and flow control device of claim 13, wherein the pump unit comprises a squeeze bulb that includes an internal spring to aid in re-expanding the squeeze bulb when external foot pressure is removed.

16. The foot operated irrigation pump and flow control device of claim 13, wherein the flow control unit and the pump unit are adapted to minimally or not at all impede with fluid flow therethrough when the flow control unit is in an open position.
